(19) United States
(12) Patent Application Publication
(10) Pub. No.: US 2022/0339358 A1
(43) Pub. Date: Oct. 27, 2022

(54) AUTO-INJECTOR

(71) Applicant: Owen Mumford Limited, Woodstock, Oxfordshire (GB)

(72) Inventor: Blount DAVID, Woodstock, Oxfordshire (GB)

(21) Appl. No.: 17/763,821

(22) PCT Filed: Sep. 23, 2020
(86) PCT No.: PCT/EP2020/076503

§ 371 Date: Mar. 25, 2022

(30) Foreign Application Priority Data
Sep. 27, 2019 (GB) 1914009.4

(51) Int. Cl. A61M 5/20

(52) U.S. Cl. CPC A61M 5/2033; A61M 2005/202; A61M 2005/206; A61M 2005/208

(57) ABSTRACT

An auto-injector for receiving and operating a syringe, the auto-injector comprising: a housing for receiving the syringe. The housing comprise a main body and a door operable between a first and a second position. The syringe is receivable within the housing when the door is in the first position. The auto-injector comprises at least one biaser configured to bias the door from the first position towards the second position, or from the second position towards the first position. The auto-injector comprises a plunger driver being configured, on activation of the auto-injector, to drive a plunger forward within the auto-injector to operate the syringe received within the auto-injector, the plunger driver being further configured to be primed when the door is moved in the direction that the biaser biases the door.

### TECHNICAL FIELD

The invention relates to auto-injectors for use with syringes. The invention may relate to, but need not be limited to, safety auto-injectors and/or auto-injectors for use with safety syringes.

### BACKGROUND

Safety syringes typically include some form of safety mechanism to protect healthcare workers from a hypodermic needle of the syringe after it has been injected into a patient. Exemplary safety syringes may include a sheath for covering the needle after use of the syringe. Other exemplary syringes may cause the needle to retract within the barrel of the syringe.

Safety syringes may be broadly split into ‘active’ and ‘passive’ safety syringes. Active safety syringes typically require some action by a user of the syringe to engage the safety mechanism and/or deploy the sheath. Such action may be taken after removal of the needle from the patient, or may be taken during removal of the needle from the patient. Passive safety syringes typically engage the safety mechanism and/or deploy the sheath without any specific action by the user, that is, without any action other than that usually taken to use the syringe.

An auto-injector is a device for receiving a syringe and for driving a syringe plunger of the syringe into a barrel of the syringe without any force being applied by the user. Typically, an auto-injector includes a plunger driver and a drive spring that are arranged to provide a force to drive the syringe plunger into the barrel. The drive spring and plunger driver may be activated by operation of a button or other release mechanism on the auto-injector. A safety auto-injector may be one which includes a shroud that may be deployed to a position covering a needle of a syringe received within the auto-injector before and after use of the syringe. The shroud of the auto-injector may be deployed under a force applied by a shroud spring.

The force applied by a drive spring typically depends on the viscosity of the drug in the barrel. The more viscous a drug is, the greater the force the drive spring has to apply upon firing. The manual priming of an auto-injector with a high force drive spring can therefore be challenging if the force required to prime it is too high for a typical user of the auto-injector.

An improved auto-injector and method for priming thereof is required.

### SUMMARY

According to aspect of the invention there is provided an auto-injector for receiving and operating a syringe, the auto-injector comprising: a housing for receiving the syringe, the housing comprising a main body and a door operable between a first and a second position, wherein the syringe is receivable within the housing when the door is in the first position; at least one biaser configured to bias the door from the first position towards the second position, or from the second position towards the first position; and a plunger driver being configured, on activation of the auto-injector, to drive a plunger forward within the auto-injector to operate the syringe received within the auto-injector, the plunger driver being further configured to be primed when the door is moved in the direction that the biaser biases the door.

The biaser assists a user in the priming of an auto-injector by providing an assisting force.

Optionally, the plunger driver comprises one or more first springs.

Optionally, the or each of the one or more first springs comprises a tension spring.

Optionally, the biaser comprises one or more second springs.

Optionally, the main body and the door are connected by a hinge, and the auto-injector is provided with a charging link between the main body and the door wherein the connection of the charging link to the main body and/or the connection of the charging link to the door is a slidable connection configured to slide when the door moves between the first and second positions, the charging link being configured to couple to a plunger driver for priming thereof on a movement of the door.

Optionally, the or each of the one or more second springs comprises a torsion spring.

Optionally, the torsion spring is coupled to the door and the main body.

Optionally, the main body and the door are configured to twist the torsion spring on an opening or closing movement of the door.

Optionally, the torsion spring is configured to apply a torque at the hinge of the door to bias the door.

Optionally, the or each of the one or more second springs comprises a tension spring and/or an compression spring.

Optionally, the main body and the door are connected by a sliding means, and the auto-injector is provided with a charging link between the main body and the door wherein the connection of the charging link to the main body and/or the connection of the charging link to the door is a slidable connection configured to slide when the door moves between the first and second positions, the charging link being configured to couple to a plunger driver for priming thereof on a movement of the door.

Optionally, the second position of the door comprises a closed position of the door.

Optionally, the movement of the door to the second position comprises the closing movement of the door.

Optionally, the connection of the charging link to the door is positionally fixed and the connection of the charging link to the main body is slidable.

Optionally, the connection of the charging link to the door is positioned at a point up to a half, up to a third, or up to a quarter of the length of the door from a hinge of the door.

Optionally, the main body and the connection of the charging link to the door are configured such that a maximum angle between the plane of the main body and the charging link during: (i) the movement of the door to the first position; and (ii) the movement of the door to the second position is up to 45 degrees.

Optionally, a driving force of the plunger driver is in the range from 30-50 Newtons.

Optionally, the charging link comprises a shuttle configured to travel along a shuttle guide to provide a slidable connection of the charging link to the main body

Optionally, the shuttle comprises a first priming portion coupled to the plunger driver and configured to travel along the shuttle guide when the door is moved in a direction opposite to that in which the biaser biases the door.

Optionally, the main body and/or the first priming portion comprises a latch configured to retain the first priming portion in position after the door has moved in a direction opposite to that in which the biaser biases the door.

Optionally, the shuttle comprises a second priming portion configured to travel along the shuttle guide in the direction that the door is biased for priming the plunger driver.

Optionally, the first and second priming portions are configured to travel together along the shuttle guide on movement of the door in the direction opposite to the direction the biaser biases the door, and are separable such that the second priming portion separates from the first priming portion and travels along the shuttle guide on the movement of the door in the direction that the door is biased.

### BRIEF DESCRIPTION OF THE DRAWINGS

Exemplary embodiments are disclosed herein with reference to the accompanying drawings, in which:

FIG. 1a-1b show perspective views of an auto-injector;

FIGS. 2a-2b show enhanced views of a biaser of an auto-injector;

FIG. 3 shows a perspective view of an auto-injector undergoing priming;

FIG. 4a shows a perspective view of an auto-injector after priming; and

FIG. 4b shows a perspective view of an auto-injector during activation.

FIGS. 5a-5d show perspective views of an auto-injector undergoing priming and firing.

### DETAILED DESCRIPTION

Generally, disclosed herein are exemplary methods and apparatus for auto-injectors. The term “auto-injector” is used herein and may be considered to encompass both an auto-injector and a safety auto-injector, as appropriate. The auto-injectors may be configured to receive and operate a standard syringe (i.e. not a safety syringe) and/or a safety syringe.

In the following embodiments, the terms “forward” and “front” refer to the patient facing end of the injection device or component thereof. In other words, the front end of the injection device is the end proximal to the injection site during use. Likewise, the term “rear” refers to the non-patient end of the injection device assembly or component thereof. In other words, the term “rear” means distant or remote from the injection site during use. Further, the term longitudinal is used to encompass a direction along or parallel to a longitudinal axis of the injection device.

Features of the exemplary arrangements disclosed herein are described as being “coupled” to other features. This term encompasses any coupling that results in the coupled features moving together in any direction, whether that be on a 1:1 basis or on some geared basis. The term “coupled” also encompasses any one of a connection between features, an abutment of one feature against another and an engagement of one feature with another, and such coupling may be direct or may be indirect, i.e. with a third feature there between.

In general terms, the invention is directed to assisting a user in the priming of an auto-injector by using a biaser providing an assisting force. A user may prime a plunger driver of an auto-injector by applying a force to the plunger driver. This may be done, for example by opening or closing a door of the auto-injector. The biaser may encompass any mechanism that applies a force in a direction assisting a user when the user is applying a force to prime a plunger driver. The biaser may also be termed a priming assist or priming assistance mechanism. An auto-injector may be considered primed when the plunger driver is in a configuration suitable to move the plunger of a syringe and thereby deliver medication using the auto-injector.

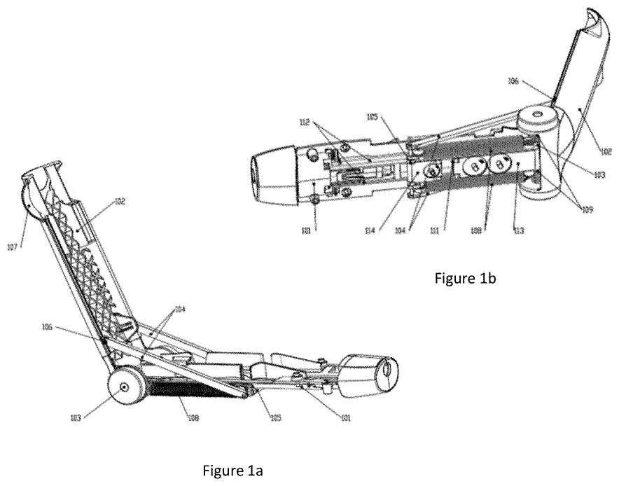

FIGS. 1a and 1b show perspective views of an exemplary auto-injector for receiving and operating a syringe (not shown). The exemplary auto-injector is shown in an open position.

The auto-injector 100 comprises a housing that further comprises a plurality of component parts. In the configuration of FIG. 1a, the housing comprises a main body 101 and a hinged door 102. The hinged door 102 is operable into a primed position from an unprimed position. The hinged door 102 is in an unprimed position in FIGS. 1a and 1b. That is, in the exemplary configuration of FIGS. 1a and 1 b, the open position of the hinged door 102 is the unprimed position and the closed position is the primed position. In other exemplary arrangements, the unprimed position may be when the hinged door 102 is closed, or at any point between closed and fully open. For the remainder of this description, the hinged door 102 is considered to be open when in the unprimed position.

A syringe (not shown) is receivable within the housing, for example, in the main body 101, when the hinged door 102 is in the unprimed position. The main body 101 and hinged door 102 are connected at a hinge 103. The door 102 is therefore rotatable relative to the main body 101. In FIGS. 1a and 1 b, the hinge is located at the rear end of the main body 101 and the hinged door 102. In other arrangements, the hinge 103 may be positioned at locations further forward on one or both of the hinged door 102 and the main body 101.

At least one charging link 104 is connected between the main body 101 and the hinged door 102. In the example of FIGS. 1a and 1b, two charging links 104 are shown. A connection 105 of the charging link 104 to the main body 101 is a slideable connection configured to slide on movement of the hinged door 102 between its open and closed positions and thus between its primed and unprimed positions. A connection 106 between the hinged door 102 and the charging link 104 is fixed with respect to its position on the hinged door 102. The connection 106 between the charging link 104 and the hinged door 102 is rotatable. The connection 106 is at a point on the hinged door 102 where, when the hinged door 102 is open, the connection 106 is rearward of the hinge 103 and when the hinged door 102 is closed the connection 106 is forward of the hinge 103 i.e. the connection 106 rotates about the hinge during opening and closing of the hinged door 106. In other arrangements, the connection 106 may be slidable and/or the connection 105 may be positionally fixed.

Optionally, the hinged door 102 comprises gripping features, which in the example shown comprise ergonometric handles 107. The gripping features may include any features that allow greater purchase for a user when pulling the hinged door 102 open and/or pushing the hinged door 102 closed. For example, the gripping features might include any type of handle, lip, flange or gripping surface for a user to open and/or close the hinged door 102.

The auto-injector 100 further comprises a plunger driver 108. The plunger driver 108 is configured to drive a plunger of a syringe received within the auto-injector 100 forwards to dispense a fluid from the syringe. In the exemplary arrangement shown in FIGS. 1a and 1 b, the plunger driver comprises at least one spring or other biasing member, which may be a tension spring, a compression spring or a torsion spring but in the example shown comprises tension springs. The exemplary plunger driver 108 shown comprises two springs. The two springs are tension springs in the example shown.

The auto-injector 100 further comprises at least one biaser 109 configured to bias the hinged door 102 towards the primed position. As described below, the biaser 109 assists the priming of the plunger driver 108 of the auto-injector 100 by providing an assisting force to help a user move the hinged door 102 to its primed position, ready for firing.

The biaser 109 may comprise one or more springs, which may be tension springs, compression springs, torsion springs or other types of spring. In the example of FIGS. 1a and 1 b, the biaser 109 comprises two torsion springs coupled to the hinged door 102 and to the main body 101 about the hinge 103. Relative movement between the hinged door 102 and the main body 101 about the hinge 103 twists the torsion springs on opening and/or closing movement of the hinged door 102. In the example of FIGS. 1a and 1 b, opening the hinged door 102 to load a syringe into the auto-injector 100 primes the torsion springs. The torsion springs 109 are primed when the hinged door 102 is in the unprimed position. The torsion springs act on the hinged door 102 to apply a torque to bias the hinged door 102 towards its primed position (in this case the closed position).

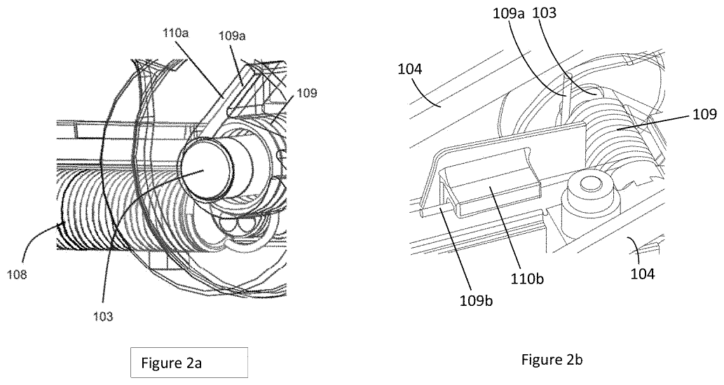

FIGS. 2a and 2b show enlarged views of the torsion springs 109, in the configuration where the biaser comprises torsion springs 109 at the hinge 103 between the hinged door 102 and main body 101. A first end 109a of the torsion spring 109 may be coupled to the hinged door 102. The first end 109a may be coupled by engaging with a pocket or shelf 110a formed in, formed on, or connected to the hinged door 102. A second end 109b of the torsion spring 109 may be coupled to the main body 101. The second end 109b may be coupled by engaging with a pocket or shelf 110b formed in, formed on, or connected to the main body 101. The torsion spring 109 comprises a helical structure between the first and second ends 109a, 109b. Opening of the helical structure by relative rotation of the first and second ends 109a, 109b stores energy within the torsion spring, which is therefore biased towards a tightening of the helical structure. In the exemplary arrangement of FIGS. 2a and 2b, the torsion spring is biased towards closing the hinged door 102 and therefore into the primed position.

FIGS. 2a and 2b show only one torsion spring and the arrangement shown and described above may be replicated for the torsion spring on the opposite side of the auto-injector 100.

The connection 105 is configured to couple to the plunger driver 108 for priming thereof on a priming movement of the hinged door 102. That is, in the example shown in FIGS. 1a and 1 b, on closing the hinged door the slidable connection 105 slides forward along the main body 101, thereby tensioning the tension springs of the plunger driver 108. The plunger driver 108 may comprise a spring or a plurality of springs.

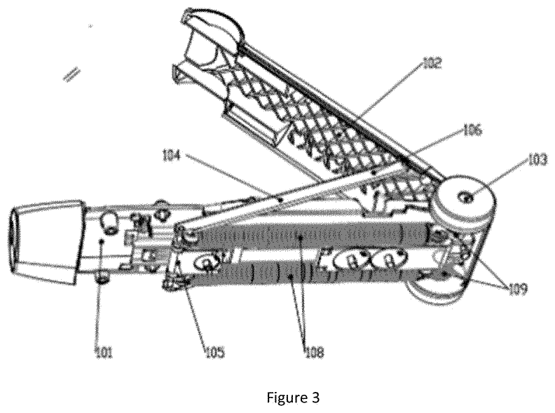

In the example of FIGS. 1a and 1 b, opening the hinged door 102 translates the plunger driver 108 rearwardly without priming it. The opening movement of the hinged door 102 thus permits a syringe to be loaded into the auto-injector 100 before priming. Before closing of the hinged door 102, an end of the tension springs of the plunger driver is retained in position relative to the main body 101. On closing the hinged door 102, through its connection 106 the charging link 104 is pushed in a forwards direction. In turn, the charging link 104, through its slidable connection 105 to the main body 101 and because it is coupled to an opposite end of the tension springs, slides along the main body 101 and thereby extends the tension springs of the plunger driver 108, as is shown later in FIG. 3.

The charging link 104, through its connections 105, 106 to the main body 101 and hinged door 102 converts the arc shaped movement of the hinged door 102 as it opens into a linear force (in this example in a forwards direction) on the plunger driver 108 to prime it.

The positioning of the connection 106 on the hinged door 102 and/or the length of the charging link 104 determines a level of mechanical advantage provided to a user by this mechanism. It is therefore easier for a user to prime the plunger driver 108 during the priming movement of the hinged door. The longer the charging link 104 and/or the closer the connection 106 is positioned to the hinge 103, the shallower the maximum angle the charging link 104 forms to the main body 101 during the priming movement of the hinged door 102. A shallower angle results in a greater mechanical advantage and allows the plunger driver 108 to be primed more easily. This permits higher force springs to be used in the plunger driver 108. It is noted, however, that there is a trade-off between having a shallow angle to increase mechanical advantage and having a steeper angle to increase the amount of forward translation of the connection 105, but a decreased mechanical advantage.

Thus, in exemplary arrangements the connection 106 may be positioned, for example, at a point up to half, up to a third, up to a quarter, or up to a smaller fraction of the length of the hinged door 102 from a hinge of the hinged door 102.

In exemplary arrangements, the main body 101, the location of the connection 106 and/or the hinged door 102 are configured such that a maximum angle between the plane of the main body 101 and the charging link 104 during the opening movement and/or closing movement of the hinged door 102 is up to 45 degrees. Typically, the maximum angle of the charging link 104 will be achieved when the hinged door 102 is perpendicular to the main body 101. Other maximum angles are possible, for example, the maximum angle may be up to 35 degrees, 25 degrees or 15 degrees.

FIG. 3 shows a perspective view of the auto-injector 100 during priming. As described above, the priming movement of the hinged door 102, through its connection 106 to the charging link 104, pushes a forward end of the charging link 104 in a forwards direction. In turn, the charging link 104, through its slidable connection 105 to the main body 101, slides along the main body 101 and thereby primes the plunger driver 108. The biaser 109 is configured to assist in moving the hinged door 102 to the primed position. More specifically, the torsion springs of the biaser 109, which have been primed during opening of the hinged door 102, exert a rotational force on the hinged door 102 urging it to a primed (in this case closed) position. This assists the user in priming the plunger driver 108. Put another way, the torsion springs are configured to store energy delivered by a user when opening the hinged door 102 and then to release the stored energy to assist the user in closing the hinged door 102.

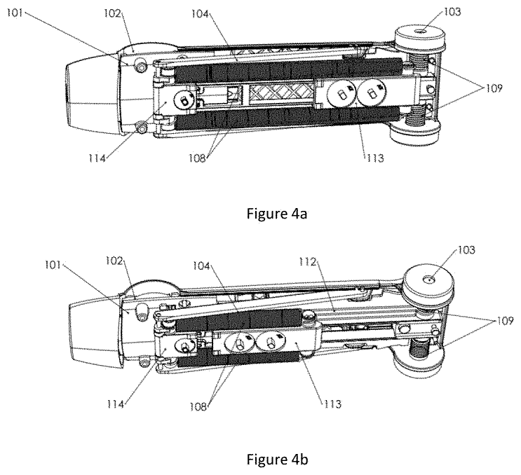

FIG. 4a shows a view of the underside of the auto-injector 100 in a primed state with the hinged door 102 in its primed position and the plunger driver 108 primed. The plunger driver 108 is retained in its primed state by the charging link 104, the position of which is maintained by the primed position of the hinged door 102. In the example of FIG. 4a, the two tension springs are fully extended.

FIG. 4b shows a perspective view of the underside of the auto-injector 100 during activation. The plunger driver 108 is configured, on activation to drive a plunger forward within the auto-injector 100 to operate the syringe received with the auto-injector 100. In the example of FIG. 4b, the tension springs of the plunger driver 108 work together to provide the forward force necessary to cause a drug in the mounted syringe to be injected into a patient. The plunger may be coupled to the plunger driver 108 to achieve this. In contrast to known devices whose maximum drive spring strength is typically limited to the force a user can reasonably apply during unassisted priming movement, the present invention comprises the biaser 109 that provides an assisting force during priming. In this way the plunger driver 108 can comprise a high force spring and can therefore provide a higher driving force during operation. For example, the plunger driver may be able to provide a driving force to the plunger in a range from 30-50 Newtons. In some arrangements, a maximum force a user needs to apply at any point to prime the plunger driver 108 may be low, for example in a range from 3-25 Newtons. Preferably the maximum force a user needs to apply at any point to prime the plunger driver is between 3 and 7 Newtons, and even more preferably the force a user needs to apply at any point to prime the plunger drivers is 5 Newtons.

Further optional features and components will now be described with reference to FIGS. 1b, 4a, and 4b.

The charging link 104 may be coupled to a shuttle 111. The shuttle 111 may be slidable along the main body 101 and configured to travel along a shuttle guide 112. The connection 106 may be between the charging link 104 and the shuttle 111 and may be rotatable. In some arrangements, the shuttle 111 provides the slideable connection of the charging link 104 to the main body 101.

The shuttle 111 comprises a first priming portion 113 and a second priming portion 114. The tension springs of the plunger driver 108 are connected between the first and second priming portions 113, 114. The first and second priming portions 113, 114 are configured to travel along the shuttle guide 112 together towards the hinge 103 on opening of the hinged door 102. As the tension springs are connected between the first and second priming portions, this movement does not prime the plunger driver 108.

The main body 101 and/or the first priming portion 113 may comprise a latch configured to retain the first priming portion 113 in position on the shuttle guide 112 after opening of the hinged door 102. The first and second priming portions 113, 114 are separable after opening of the hinged door 102. The second priming portion 114 is configured to travel along the shuttle guide 112 away from the hinge 103 on closing of the hinged door 102. Because the tensions springs are connected between the first and second priming portions 113, 114, as the second priming portion 114 travels forward the tension springs of the plunger driver 108 are primed.

As described above, the first and second priming portions 113, 114 are configured to travel together along the shuttle guide 112 on opening of the hinged door 102, and are separable such that the second priming portion 114 separates from the first priming portion 113 and travels along the shuttle guide 112 on closing of the hinged door 102. This separated state is shown in FIG. 4a.

Upon firing, caused for example by pressing a front end of the auto-injector 100 on a patient's skin, the first priming portion 113 is released from its latch and travels forward along the shuttle guide 112 under the force of the plunger driver 108. In the example of FIGS. 4a and 4b, the tension springs pull the first priming portion 113 of the shuttle 111 in a forward direction. As shown in FIGS. 4a and 4b, the shuttle guide 112 is coupled to plunger driver 108 such that movement thereof follows a path determined by the shuttle guide 112. The first priming portion 113 is coupled to a syringe plunger of the syringe and therefore drives it forwards into a barrel of the syringe.

The auto-injector 100 may also comprise a shroud at least partially covering and extending forwards beyond a forward end of a needle of the syringe when the syringe is fitted within the auto-injector and before use. When present, the shroud may be configured, on rearward movement thereof, for example caused by pressing of the shroud onto a patient's skin, to release the latch and fire the device. Other firing mechanisms envisaged include side or rear buttons.

The auto-injector 100 may also comprise a ratchet operable during the closing movement of the hinged door 102 to prevent movement of the hinged door in the opening direction by a force exerted by the plunger driver 108. With reference to FIG. 3, as the door 102 is moved to its closed position and the plunger driver 108 becomes primed, the plunger driver 108 exerts an opposing force against the closing hinged door 102. If a user does not maintain a force on the hinged door 102 during closing before it is fully closed, it will spring back open. The ratchet prevents this from happening by permitting movement of the hinged door 102 in a closing direction but preventing it in the opening direction. The ratchet is configured to become operable only after the user has opened the door for the first time so that it does not interfere with a user's ability to open the hinged door 102 for to load the syringe into the main body 102.

The skilled person will be able to envisage other assemblies, auto-injectors and features thereof without departing from the scope of the appended claims. In particular, it is noted that one or more features included in one or more drawings may be integrated into auto-injectors shown in other drawings, as will be appreciated by the skilled person.

For example, whilst the biaser 109 has been described above to comprise one or more torsion springs around a hinge 103, it may additionally and/or alternatively comprise one or more tension and/or compression springs. As with the torsion spring, a tension or compression spring may assist a user in the priming (e.g. closing) movement of the hinged door 102 by biasing the hinged door 102 towards its primed position. In one example, a first end of a compression spring may be connected to the main body 101 rearwards of the shuttle 112. This connection of the first end to the main body 101 is positionally fixed. A second end is coupled directly or indirectly to the charging link 104. This may comprise a connection to the shuttle 112. As the hinged door 102 is opened, the charging link 104 slides rearward compressing the compression spring. The compression spring then exerts a force on the charging link 104 to bias it towards the front end of the device and thus also bias the hinged door 102 to its primed, closed position. During the priming movement of the hinged door 102 (e.g. the closing movement), the additional force in the forward direction provided by the compression spring reduces the force a user needs to apply to prime the auto-injector 100. The arrangement above may be adapted to accommodate a tension spring by connecting the first end of the spring to the main body 101 at a position forwards of the shuttle 112. The tension spring is therefore extended on opening of the door 102.

In another example, the auto-injector may be configured such that the biaser 109 comprising either torsion springs or one or more tension and/or compression springs is configured to bias the door towards an open position. In such a configuration the priming movement of the door is the opening of the door. The plunger driver of such an auto-injector may be anchored at a first end away from the hinge of the auto-injector. The second priming portion 114 is configured to travel along the shuttle guide 112 together towards the hinge 103 on opening of the hinged door 102. As the tension spring is anchored away from the hinge end this movement primes the plunger driver 108. The main body 101 and/or the first priming portion 113 may comprise a latch configured to retain the first priming portion 113 in position on the shuttle guide 112 after opening of the hinged door 102.

Upon firing, caused for example by pressing a front end of the auto-injector 100 on a patient's skin, the first priming portion 113 is released from its latch and travels forward along the shuttle guide 112 under the force of the plunger driver 108. As described previously the tension springs pull the first priming portion 113 of the shuttle 111 in a forward direction. The shuttle guide 112 is coupled to plunger driver 108 such that movement thereof follows a path determined by the shuttle guide 112. The first priming portion 113 is coupled to a syringe plunger of the syringe and therefore drives it forwards into a barrel of the syringe.

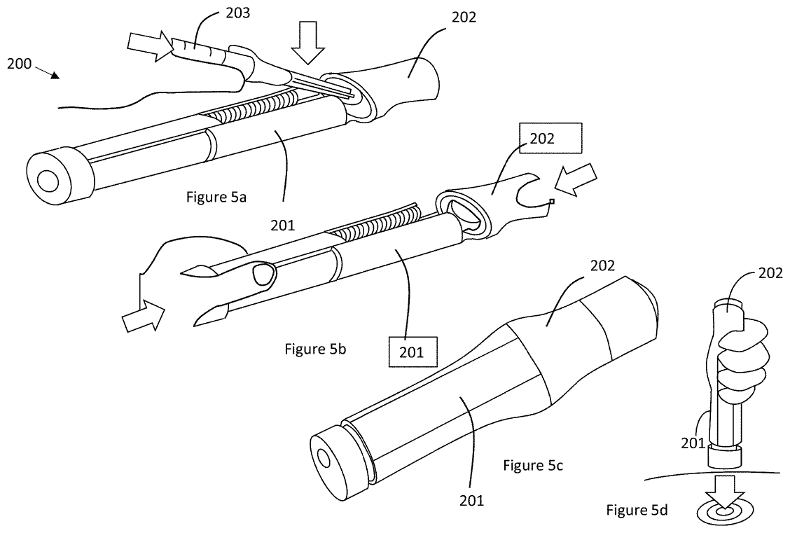

Although the present invention has been described with reference to a hinged door it may be understood that other door configurations are possible. For example the main body and door may have a slideable connection as illustrated in the example auto-injector 200 of FIGS. 5a to 5d. Such an auto-injector 200 will function in the same way as described previously with reference to FIGS. 1 to 4. The main body 201 and door 202 may be connected by any suitable means, for example a projection from the door 202 may be received within the main body 201. As was the case in the example configurations of FIGS. 1 to 4, a charging link between the main body and door may be provided. The charging link may be configured to slide when the door 202 is moved between the first and second positions as shown in the transition between FIGS. 5b and 5c. The charging link may be configured to couple to the plunger driver of the auto-injector 200 for priming thereof on a movement of the door. As in the examples of FIGS. 1 to 4, the biaser is configured to bias the door from the first position towards the second position, or from the second position towards the first position. In either case, the biaser may comprise a spring, for example a tension or compression spring.

In the example configurations of FIGS. 5a-5d, the first position of the auto-injector is the open position and a syringe 203 may be loaded into the auto-injector 200 when it is in its open position. After the syringe 203 is loaded, the door may be moved towards its second position, in this case the closed position. The auto-injector may then be fired by pressing a front end onto the surface of a user's skin, or by a different firing mechanism such as a button. It is envisaged that the plunger driver of the example auto-injector of FIGS. 5a-5d may be primed on either or both the opening or closing movements of the door and the biaser is configured to assist in the priming of the plunger driver accordingly. It will be appreciated that the advantages provided by the biaser as described herein are realised in all such configurations.

1. An auto-injector for receiving and operating a syringe, the auto-injector comprising:
a housing for receiving the syringe, the housing comprising a main body and a door operable between a first and a second position, wherein the syringe is receivable within the housing when the door is in the first position;
at least one biaser configured to bias the door from the first position towards the second position, or from the second position towards the first position; and
a plunger driver being configured, on activation of the auto-injector, to drive a plunger forward within the auto-injector to operate the syringe received within the auto-injector, the plunger driver being further configured to be primed when the door is moved in the direction that the biaser biases the door.

2. An auto-injector according to claim 1, wherein the plunger driver comprises one or more first springs.

3. An auto-injector according to claim 2, wherein the or each of the one or more first springs comprises a tension spring.

4. An auto-injector according to claim 1, wherein the biaser comprises one or more second springs.

5. An auto-injector according to claim 4, wherein the main body and the door are connected by a hinge, and the auto-injector is provided with a charging link between the main body and the door wherein the connection of the charging link to the main body and/or the connection of the charging link to the door is a slidable connection configured to slide when the door moves between the first and second positions, the charging link being configured to couple to a plunger driver for priming thereof on a movement of the door.

6. An auto-injector according to claim 5, wherein each of the one or more second springs comprises a torsion spring.

7. An auto-injector according to claim 6, wherein the torsion spring is coupled to the door and the main body.

8. An auto-injector according to claim 7, wherein the main body and the door are configured to twist the torsion spring on an opening or closing movement of the door.

9. An auto-injector according to claim 6, wherein the torsion spring is configured to apply a torque at the hinge of the door to bias the door.

10. An auto-injector according to claim 4, wherein the or each of the one or more second springs comprises a tension spring and/or a compression spring.

11. An auto-injector according to claim 10, wherein the main body and the door are connected by a sliding means, and the auto-injector is provided with a charging link between the main body and the door wherein the connection of the charging link to the main body and/or the connection of the charging link to the door is a slidable connection configured to slide when the door moves between the first and second positions, the charging link being configured to couple to a plunger driver for priming thereof on a movement of the door.

12. An auto-injector according to claim 1, wherein the second position of the door comprises a closed position of the door.

13. An auto-injector according to claim 1, wherein the movement of the door to the second position comprises the closing movement of the door.

14. An auto-injector according to claim 5, wherein the connection of the charging link to the door is positionally fixed and the connection of the charging link to the main body is slidable.

15. An auto-injector according to claim 5, wherein the connection of the charging link to the door is positioned at a point up to a half, up to a third, or up to a quarter of the length of the door from a hinge of the door.

16. An auto-injector according to claim 5, wherein the main body and the connection of the charging link to the door are configured such that a maximum angle between the plane of the main body and the charging link during:
(i) the movement of the door to the first position; and
(ii) the movement of the door to the second position is up to 45 degrees.

17. An auto-injector according to claim 1, wherein a driving force of the plunger driver is in the range from 30-50 Newtons.

18. An auto-injector according to claim 5, wherein the charging link comprises a shuttle configured to travel along a shuttle guide to provide a slidable connection of the charging link to the main body

19. An auto-injector according to claim 18, wherein the shuttle comprises a first priming portion coupled to the plunger driver and configured to travel along the shuttle guide when the door is moved in a direction opposite to that in which the biaser biases the door.

20. An auto-injector according to claim 19, wherein the main body and/or the first priming portion comprises a latch configured to retain the first priming portion in position after the door has moved in a direction opposite to that in which the biaser biases the door.

21. An auto-injector according to claim 19, wherein the shuttle comprises a second priming portion configured to travel along the shuttle guide in the direction that the door is biased for priming the plunger driver.

22. An auto-injector according to claim 21, wherein the first and second priming portions are configured to travel together along the shuttle guide on movement of the door in the direction opposite to the direction the biaser biases the door, and are separable such that the second priming portion separates from the first priming portion and travels along the shuttle guide on the movement of the door in the direction that the door is biased.
